(19) United States
(12) Patent Application Publication
(10) Pub. No.: US 2025/0064637 A1
(43) Pub. Date: Feb. 27, 2025

(54) ANTI-VACUUM SYSTEM FOR A PHACOEMULSIFIER

(71) Applicant: Johnson & Johnson Surgical Vision, Inc., Irvine, CA (US)

(72) Inventor: Assaf Govari, Haifa (IL)

(21) Appl. No.: 18/789,075

(22) Filed: Jul. 30, 2024

Related U.S. Application Data
(60) Provisional application No. 63/534,156, filed on Aug. 23, 2023.

(51) Int. Cl. A61F 9/007

(52) U.S. Cl. CPC A61F 9/00745

(57) ABSTRACT

A phacoemulsification valve for controlling flow of eye fluid, consisting of an enclosure, a first lumen within the enclosure, having a first and a second termination, a second lumen within the enclosure, intersecting the first lumen, so that the first lumen separates the second lumen into a first and a second section, and a pin, that slides within the first lumen between a first position, wherein the pin prevents transfer of the eye fluid between the first and second sections and a second position, wherein the pin permits transfer of the eye fluid between the first and second sections. The valve also includes a first coil, proximate to the first termination, which when energized transfers the pin between the first and second positions, and a second coil, positioned in proximity to the second termination, which when energized transfers the pin between the second and first positions.

### CROSS-REFERENCE TO RELATED APPLICATION

This application claims the benefit of U.S. Provisional Patent Application 63/534,156, filed Aug. 23, 2023, which is incorporated herein by reference.

### FIELD OF THE DISCLOSURE

This disclosure relates generally to ocular surgery, and specifically to operation of a phacoemulsifier used for the surgery.

### BACKGROUND

A cataract is a cloudy area in the lens of the eye that leads to a decrease in vision. Phacoemulsification is a modern cataract surgery method in which the eye's internal lens is emulsified with an ultrasonic handpiece and aspirated from the eye. The aspiration needs to be controlled.

### BRIEF DESCRIPTION OF THE DRAWINGS

The present disclosure will be understood from the following detailed description, taken in conjunction with the drawings in which:

FIG. 1 is a pictorial view of a phacoemulsification apparatus, according to an example of the present disclosure;

FIGS. 2A and 2B are schematic cross-sections of an anti-vacuum surge system (AVSS) used in the apparatus of FIG. 1, according to an example of the present disclosure;

FIG. 3 is a pictorial view of a phacoemulsification apparatus, according to an alternative example of the present disclosure;

FIGS. 4A and 4B are schematic cross-sections of an AVSS used in the apparatus of FIG. 3, according to an example of the present disclosure; and

FIGS. 5A, 5B, 5C, and 5D are schematic cross-sections of an AVSS used in the apparatus of FIG. 3, according to a further alternative example of the present disclosure.

### DESCRIPTION OF EXAMPLES

### Overview

Phacoemulsification is a procedure where the natural lens of a patient's eye is emulsified, and the emulsified matter is then aspirated from the eye. (Following the phacoemulsification procedure an artificial lens is inserted into the eye.) The phacoemulsification procedure may be performed by a physician using a handpiece to which a hollow needle is attached. The physician inserts the hollow needle into the patient's eye lens, and vibrates the distal end of the needle ultrasonically to emulsify the lens (typically the needle may first be used to divide the lens into portions). Emulsified matter and irrigation fluid (collectively, “eye fluid”) are then aspirated from the eye using an aspiration pump connected with the hollow needle.

The aspiration of the eye fluid is implemented using an aspiration pump, which applies a vacuum, via an aspiration tubing line and an aspiration channel in the handpiece, to the hollow needle. However, during the procedure some lens material may at least partially block the distal end of the needle, so that the vacuum, increases within the aspiration tubing line, i.e., the vacuum pressure becomes lower. If the blockage releases, the vacuum built up in the aspiration tubing line causes a “vacuum surge” or post occlusion surge such that eye fluid is quickly pulled out of the eye through the needle, and may cause significant trauma to the eye.

To prevent the vacuum surge, examples of the present disclosure use an anti-vacuum surge system (AVSS) coupled with the handpiece.

In a disclosed example, the AVSS is separate and displaced from the handpiece, and is configured to be inserted into the aspiration tubing line so that a first part of the aspiration tubing line connects the AVSS with the handpiece, and a second part of the tubing line connects the AVSS with the aspiration pump. The AVSS has a lumen that fluidly connects the aspiration channel of the handpiece to the aspiration pump via the aspiration tubing line.

A ferromagnetic pin translates in a portion of the AVSS lumen, between a first “closed” position wherein it prevents eye fluid transfer through the AVSS lumen, and a second “open” position wherein it permits the transfer. The AVSS has two coils, each formed of a single wire, which are positioned on opposite sides of a portion of the AVSS lumen, i.e., the coils “sandwich” a portion of the AVSS lumen. Energizing one of the coils with direct current (DC) maintains the pin in the open position; energizing the other coil with DC current maintains the pin in the closed position.

The AVSS receives a signal from a pressure sensor, located in the handpiece, which provides an indication of the pressure of the eye fluid in the aspiration tubing line. The AVSS also has a controller which receives the pressure indication and in response energizes one of the two coils, so that the AVSS may be in either its open or closed position. In the closed position, the vacuum generated by the aspiration pump is disconnected from the patient's eye.

In an alternate example the AVSS comprises elements, that, except as described below, have functions and are structured substantially as for the example AVSS described above. The alternate AVSS may be configured to be disposable rather than reusable, so that elements of the AVSS may not be sterilizable. In the alternate example, rather than forming the coils of the AVSS from single wires, each coil may be formed from one or more layers of a printed circuit board (PCB). For a given coil a conducting trace on each surface of the layers of the PCB may be formed as a spiral, and the spirals may be connected together by vias to form the coil.

The alternate example may be configured to be attached to the handpiece, and in some examples the pressure sensor may be incorporated in the AVSS rather than in the handpiece.

In a further alternate example, the AVSS also comprises elements, that, except as described below, have functions and are structured substantially as for the example AVSS described above. The further alternate AVSS may also be disposable, may be directly attached to the handpiece, or may be separated from the handpiece, and may also use PCB coils. In some examples the pressure sensor may be incorporated in the AVSS.

However, the pin in this example, while being conductive, is non-ferromagnetic. As such the pin is maintained in each of the two positions by friction, and is moved from position to position by inducing eddy currents in the pin.

To generate the eddy currents, the AVSS comprises two capacitors, each of which may be charged and discharged separately. Each capacitor is configured to discharge through a respective one of the PCB coils, and the varying magnetic field so produced generates eddy currents in the pin, when it is proximate to the respective coil. The coils and the open and closed positions of the pin are arranged so that if the pin is in the open position, discharging through the coil proximate to the open position generates eddy currents in the pin that repel the pin to the closed position. Similarly, if the pin is in the closed position, discharging through the coil proximate to the closed position generates eddy currents in the pin that repel the pin to the open position.

A controller in the AVSS may be configured to discharge the capacitors through their respective coils in response to a pressure indication received from the pressure sensor, so that the controller is able to switch the AVSS between its open and closed positions.

### System Description

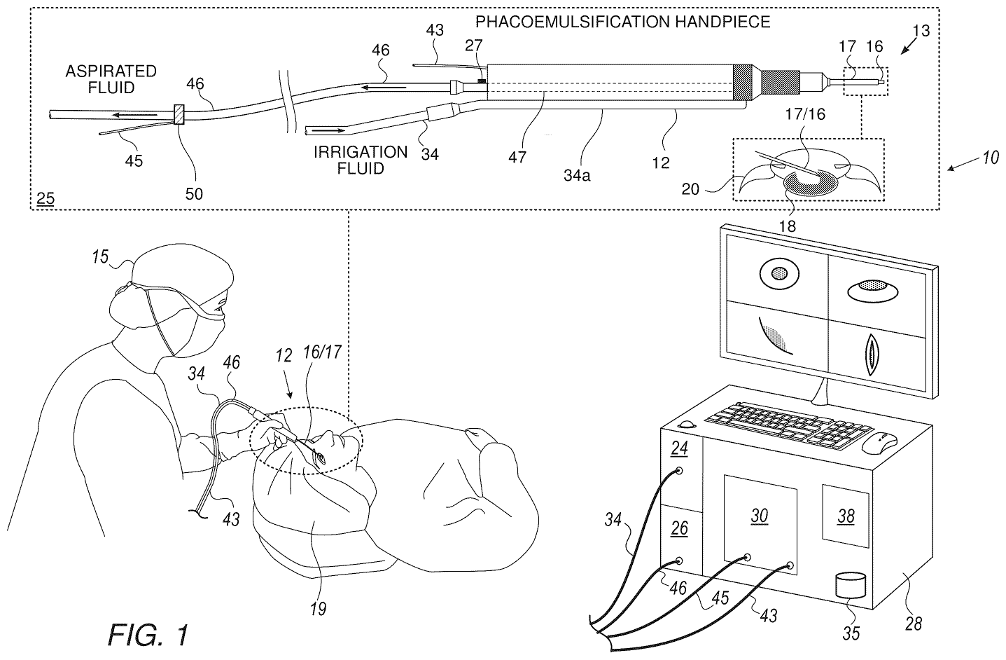

FIG. 1 is a pictorial view of a phacoemulsification apparatus 10, according to an example of the present disclosure. FIG. 1 includes an inset 25, and as shown in the figure and the inset apparatus 10 includes a phacoemulsification probe/handpiece 12 comprising a hollow needle 16 and a coaxial irrigation sleeve 17. Irrigation sleeve 17 at least partially surrounds the needle 16 and creates a fluid pathway between the external wall of the needle 16 and the internal wall of the sleeve 17. Needle 16 and sleeve 17 are also herein termed a needle-sleeve combination 13.

Needle 16 is configured to be inserted into a lens capsule 18 of an eye 20 of a patient 19. Needle 16 is mounted on a horn (not shown) of handpiece 12, and is shown in inset 25 as a straight needle. However, any suitable needle may be used with the phacoemulsification handpiece 12, for example, a curved or bent tip needle that is commercially available from Johnson & Johnson Surgical Vision, Inc, Irvine, CA, USA.

A physician 15 holds handpiece 12 so as to perform a phacoemulsification procedure on the eye 20 of patient 19. The physician may activate the handpiece using a foot pedal (not shown in the figures). Handpiece 12 comprises a piezoelectric actuator (not shown), which is configured to vibrate needle 16 and the horn upon which it is mounted in one or more vibration modes of the combined horn and needle. During the phacoemulsification procedure the vibration of needle 16 is used to break a cataract of the lens in lens capsule 18 into small pieces.

The piezoelectric actuator in the handpiece is powered by a power supply/driving module 30 in a console 28 via a cable 43. Module 30, under overall control of a processor 38 and associated memory 35 in console 28, is configured to provide power to the piezoelectric actuator as a resonant radiofrequency (RF) driving current, and is also configured to provide DC power to an anti-vacuum surge system (AVSS) 50 located between the handpiece 12 and the console 28 along aspiration tubing line 46. AVSS 50 is described further below. The power from module 30 is delivered to AVSS 50 by a cable 45. Other elements of apparatus 10 are under overall control of processor 38.

During the phacoemulsification procedure, an irrigation pump 24, which may be in or outside console 28, pumps irrigation fluid through an irrigation channel 34a in handpiece 12 to irrigation sleeve 17 so as to irrigate the eye. The fluid is pumped via an irrigation tubing line 34, running from the irrigation pump 24, that is connected with channel 34a of the probe 12.

An aspiration pump 26, which also may be located in or outside console 28, aspirates eye fluid (e.g., emulsified parts of the lens in capsule 18 and irrigation fluid), from the patient's eye via needle 16, through an aspiration channel 47 in handpiece 12. Aspiration pump 26 is coupled with and produces a vacuum within aspiration tubing line 46, aspiration channel 47 and needle 16.

Pumps 24 and 26 may be any pump known in the art (e.g., a peristaltic pump or a progressive cavity pump), and the pumps are both under overall control of processor 38. Some or all of the functions of processor 38 may be combined in a single physical component or, alternatively, implemented using multiple physical components. The physical components may comprise hard-wired or programmable devices, or a combination of the two.

An aspiration pressure sensor 27 may be located in a proximal section of handpiece 12 and coupled with the aspiration channel 47. The aspiration pressure sensor 27 provides signals, indicative of the pressure of the fluid, to processor 38, enabling the processor to control the pump rate of aspiration pump 26. In an example, aspiration sensor 27 may be located on any portion of handpiece 12 or coupled with the aspiration tubing line 46.

As explained above, the eye fluid comprises emulsified lens material, and small particles of lens material, may at least partially block the flow of the aspiration fluid out of the eye 20 because of the narrow dimensions of hollow needle 16, (the needle typically has a lumen diameter of approximately 1 mm). The blockage causes the pressure in aspiration channel 47 and aspiration tubing line 46 to decrease, i.e., the vacuum to increase (because aspiration pump 26 is operating). When the blockage clears, the vacuum present in the aspiration channel and aspiration tubing line may feed back into the eye, and this vacuum “surge” may be traumatic to the structures of the eye.

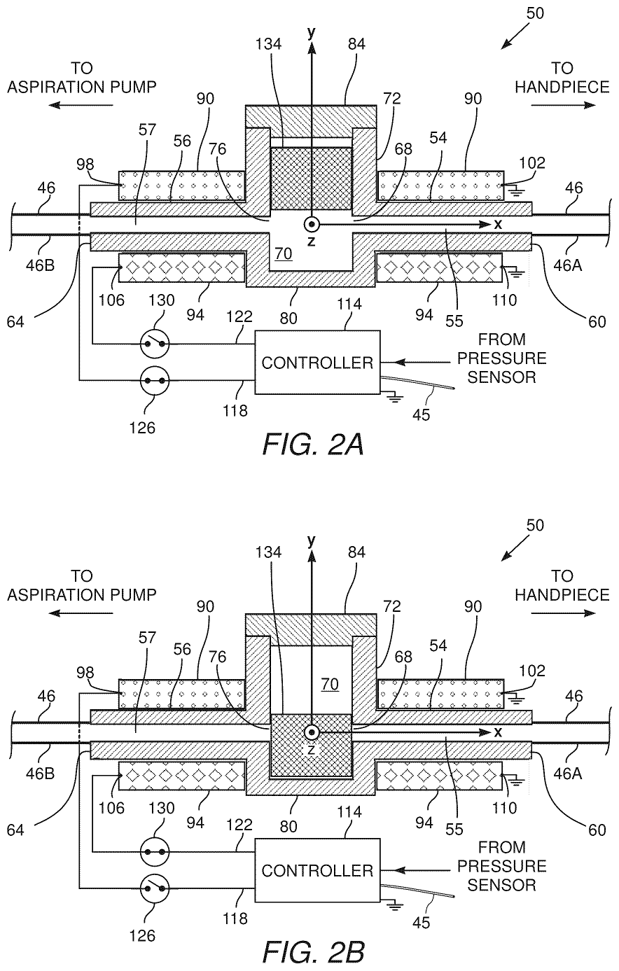

To prevent such surges, an example of the present disclosure incorporates anti-vacuum surge system (AVSS) 50 into aspiration tubing line 46. In an alternate example of the present disclosure, AVSS 50 may be incorporated into handpiece 12. At least some elements of AVSS 50 may be configured to be reusable, and AVSS 50 is described below with reference to FIGS. 2A and 2B.

### Reusable Anti-Vacuum Surge System

FIGS. 2A and 2B are schematic cross-sections of AVSS 50, according to an example of the present disclosure. AVSS 50, also herein termed valve 50, operates to open and close aspiration tubing line 46, and for clarity, the cross-sections have been drawn on a set of orthogonal xyz coordinate axes. FIG. 2A illustrates the valve 50 in an open state, wherein aspirated eye fluid may flow in aspiration tubing line 46, and FIG. 2B illustrates the valve 50 in a closed state, preventing aspirated eye fluid from flowing in the aspiration tubing line.

AVSS 50 is inserted in series with aspiration tubing line 46, cutting the line into two sections 46A, 46B, so that a proximal termination of section 46A couples with the AVSS, and a distal termination of section 46A couples with handpiece 12. Similarly, a distal termination of section 46B couples with AVSS 50, and a proximal termination of section 46B couples with module 30. Prior to performance of the phacoemulsification procedure described herein, aspiration tubing line 46 is coupled with handpiece 12, as described above, thereby enabling the signal from pressure sensor 27 to communicate with AVSS 50 via controller 114.

AVSS 50 comprises two cylindrical tubes 54, 56 having the x-axis as their common axis of symmetry; tube 54 is coupled at its distal end 60 with section 46A of the aspiration tubing line 46, and tube 56 is coupled at its proximal end 64 with section 46B of the aspiration tubing line 46. Tubes 54 and 56 define respective lumens 55 and 57. Tube 54 opens at its proximal end 68, as does lumen 55, into a cylindrical lumen 70 defined by a cylindrical valve enclosure 72, and tube 56 also opens, at its distal end 76, as does lumen 57, into lumen 70. The y-axis is the axis of symmetry of lumen 70 and cylindrical valve enclosure 72, and the xyz coordinate axes have been drawn with the origin of the coordinates midway between proximal end 68 and distal end 76.

Cylindrical valve enclosure 72 is terminated and sealed at its lower end by a fixed termination 80. Valve enclosure 72 is terminated and sealed at its upper end by a removable stopper 84. In an example of the disclosure tubes 54, 56, enclosure 72, and termination 80 may be formed, for instance by injection molding, as a single piece of plastic such as a polyimide. The type of plastic of the single piece is selected to be sterilizable so as to be reusable. Stopper 84 may also be formed from the same type of sterilizable plastic, so as to also be reusable.

A pair of planar annular coils, a coil 90 and a coil 94, are formed about cylindrical valve enclosure 72. In some examples coil 94 may be configured to be flat against termination 80. Both coils are substantially circular and have the y-axis as a common axis of symmetry. The two coils are arranged so as to “sandwich” tubes 54 and 56 and their respective lumens 55 and 57, coil 90 being above the tubes, and coil 94 being below the tubes. Each coil is formed as a spiral of a single insulated wire, so that the wire of coil 90 terminates at endpoints 98 and 102, and the wire of coil 94 terminates at endpoints 106 and 110.

A local controller 114 in AVSS 50 is configured to independently energize coils 90 and 94 with DC current via respective conducting lines 118 and 122. Each line has an independent on-off switch 126 and 130 that the controller operates. Line 118 connects to endpoint 98 of coil 90, and the other endpoint of the coil, endpoint 102, is connected to a local ground of controller 114. Line 122 connects to endpoint 106 of coil 94, and endpoint 110 of the coil is connected to a local ground of controller 114. When energized, each coil generates a magnetic field that, at the center of the coil, is in a y-direction.

AVSS 50 comprises a cylindrical ferromagnetic pin 134 in lumen 70, the pin 134 having an external radius approximately equal to the radius of lumen 70 and having the y-axis as its axis of symmetry. The height of pin 134 is larger than the diameter of tubes 54, 56. Pin 134 is configured to slide along the y-axis in lumen 70, and the pin 134 may be formed as a solid cylinder, as a hollow unfilled cylinder, or as a hollow cylinder which is filled with a non-ferromagnetic material. Other shapes of the pin 134 and lumen 70 are envisioned that would still provide the desired operation of the valve.

FIG. 2A illustrates an open state of AVSS 50, wherein upper coil 90 is energized, by controller 114 closing switch 126, while leaving switch 130 open. The magnetic field generated by the energizing of coil 90 attracts pin 134 to an “open” position in the upper section of lumen 70, so that the path between tubes 54 and 56 is open, i.e., so that aspirated eye fluid may transfer, along lumens 55 and 57, from the handpiece 12 to the aspiration pump 26.

FIG. 2B illustrates a closed state of AVSS 50, wherein lower coil 94 is energized, by controller 114 closing switch 130, while leaving switch 126 open. The magnetic field generated by the energizing of coil 94 attracts pin 134 to a “closed” position in the lower section of lumen 70, so that the path between tubes 54 and 56 is closed, i.e., so that there is no path between lumens 55 and 57. In this case aspirated eye fluid is prevented from flowing from the handpiece 12 to the aspiration pump 26, and the aspiration fluid pressure created by operation of the aspiration pump 26 no longer transfers to the handpiece 12 and eye 20.

In an example of the present disclosure, controller 114 is configured to maintain AVSS 50 in an open state as long as the pressure in the aspiration tubing line 46, as indicated to the controller by the signal from pressure sensor 27, is above a preset value or within a desired or predetermined range. When the pressure reduces below the preset value, i.e., when the vacuum in the aspiration line increases, or is outside of a desired range, indicating an at least partial blockage in the aspiration fluid flow, controller 114 is configured to change the state of AVSS 50 to a closed state, and to maintain the AVSS 50 in the closed state until the pressure in the aspiration tubing line 46, as indicated by pressure sensor 27, is above the preset value or within the desired or predetermined range.

As stated above, elements of AVSS 50 may be configured to be reusable. Thus, prior to being used in a phacoemulsification procedure, the internal elements of aspiration tubing line 46 with its attached AVSS 50, may be sterilized. The sterilization may be facilitated by removing stopper 84, enabling access to lumen 70, and to the internal lumens 55 and 57 of tubes 54 and 56 and aspiration tubing line 46. Stopper 84 may be separately sterilized, and, on removal of the stopper, pin 134 may be retrieved and sterilized, or the pin may be disposed of and replaced by a new pin.

By configuring AVSS 50 to be reusable, the wires of coils 90 and 94 may be configured to generate a stronger magnetic field than that of a disposable anti-vacuum surge system having the same overall cost, for example by increasing the number of turns of the coils and/or by reducing the resistance of the coils. The stronger magnetic field improves the reaction time of AVSS 50. Configuring pin 134, which may be relatively expensive, to be reusable, also saves cost.

While the description above of AVSS 50 has assumed it is reusable, it will be understood that other examples of AVSS 50 may be disposable. Such other examples may have a different cost and/or operate with a different magnetic field, but have the advantage of not needing to be sterilized, and those having ordinary skill in the art will be able to adapt the description, mutatis mutandis, for a disposable AVSS 50.

### Disposable Anti-Vacuum Surge System

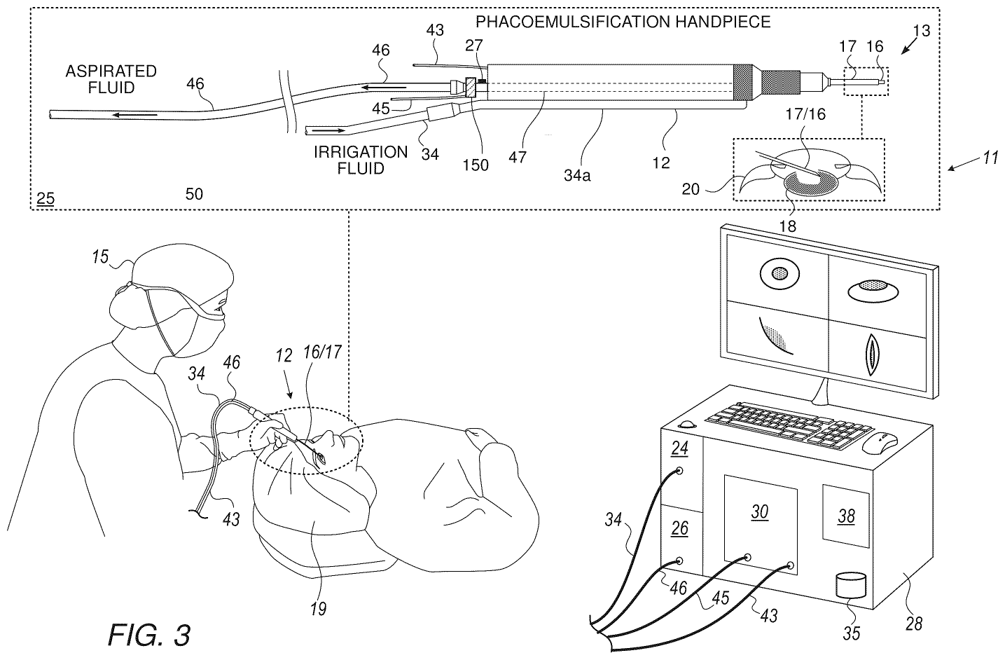

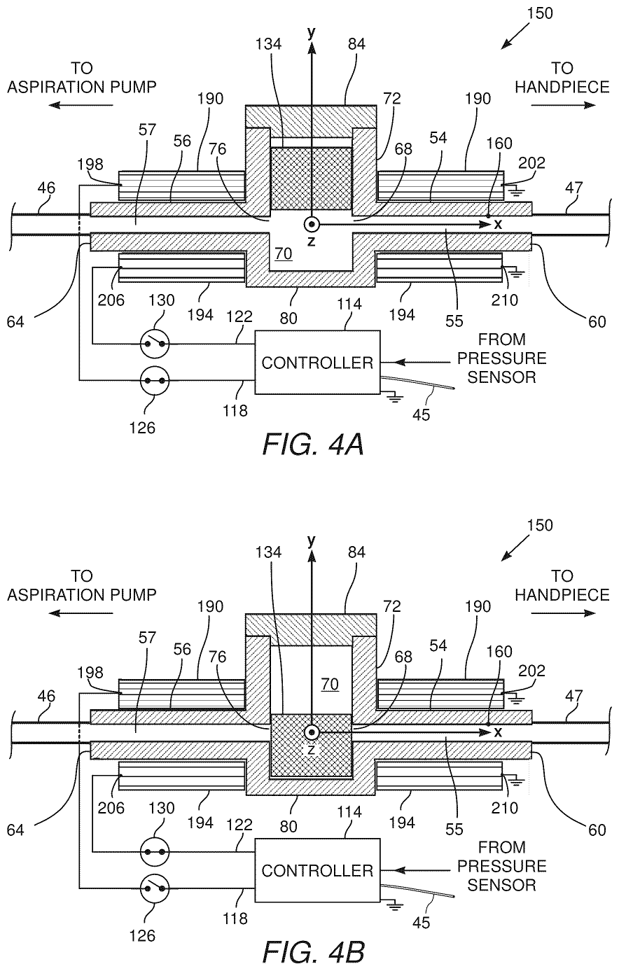

FIG. 3 is a pictorial view of a phacoemulsification apparatus 11, and FIGS. 4A and 4B are schematic cross-sections of an AVSS 150 used in apparatus 11, according to an alternative example of the present disclosure. Apart from the differences described below, the operation of apparatus 11 and AVSS 150 is generally similar to that of apparatus 10 and AVSS 50 (FIG. 1, and FIGS. 2A and 2B), and elements indicated by the same reference numerals in both apparatus 10 and apparatus 11, and AVSS 50 and AVSS 150, are generally similar in construction and in operation.

In contrast to apparatus 10, in apparatus 11, AVSS 150, also herein termed valve 150, is located at the proximal side of handpiece 12, so as to be coupled directly with channel 47, and does not separate aspiration tubing line 46 into two sections. Locating valve 150 at handpiece 12 has many benefits, including, but not limited to improving the compactness and the handling of the handpiece.

Also, in contrast to apparatus 10, in some examples of apparatus 11 pressure sensor 27 may be incorporated into AVSS 150, at a location 160 of tube 54 and lumen 55, i.e., is on the handpiece side of the AVSS, rather than being in handpiece 12.

In the following description valve 150 is assumed to be disposable, and acts to open and close aspiration tubing line 46. However, in other examples valve 150 may be configured to be reusable.

FIG. 4A illustrates AVSS 150 in an open state wherein pin 134 is in an open position, and FIG. 4B illustrates the AVSS in a closed state, wherein the pin 134 is in a closed position.

Similar to AVSS 50, AVSS 150 comprises a pair of planar annular coils, a coil 190 and a coil 194, that are formed about cylindrical valve enclosure 72, and that are substantially circular and have the y-axis as a common axis of symmetry. Coil 190 and coil 194 are arranged so as to sandwich tubes 54 and 56 and their respective lumens 55 and 57; coil 190 being above tubes 54 and 56, and coil 194 being below tubes 54 and 56.

However, in contrast to AVSS 50, each of the coils 190 and 194 of AVSS 150 are not formed of a single wire. Rather, each coil of AVSS 150 is formed from one or more layers of a printed circuit board (PCB). Conductive traces may be formed on the surfaces of each of the layers and connected together, for example by vias through the layers, so that the connected traces form one continuous galvanic three-dimensional (3D) trace. Each connected trace is spiral in form, so that the connected trace of coil 190 terminates at endpoints 198 and 202, and the connected trace of coil 194 terminates at endpoints 206 and 210.

In some examples the PCB may be flexible. In an alternative example, at least one of coils 190 and 194 may be a flat coil embedded in a PCB.

Similar to AVSS 50, local controller 114 in AVSS 150 is configured to independently energize coils 190 and 194 with DC current via respective conducting lines 118 and 122 and switches 126 and 130. Line 118 connects to endpoint 198 of coil 190, and endpoint 202 is connected to the local ground of controller 114. Line 122 connects to endpoint 206 of coil 194, and endpoint 210 of the coil is connected to the controller local ground.

Similar to AVSS 50, controller 114 is configured to maintain AVSS 150 in an open state as long as the pressure in the aspiration tubing line 46, as indicated to the controller by the signal from pressure sensor 27, is above a preset value or within a desired or predetermined range. When the pressure reduces below the preset value, i.e., when the vacuum in the aspiration line increases, or is outside of a desired range, indicating an at least partial blockage in the aspiration fluid flow, controller 114 is configured to change the state of AVSS 150 to a closed state, and to maintain the AVSS 150 in the closed state until the pressure in the aspiration tubing line 46, as indicated by pressure sensor 27, is above the preset value or within the desired or predetermined range.

As stated above, AVSS 150 is configured to be disposable, so that some or all elements of the AVSS 150, such as, for example, pin 134, coils 190 and 194, and controller 114, may be non-sterilizable.

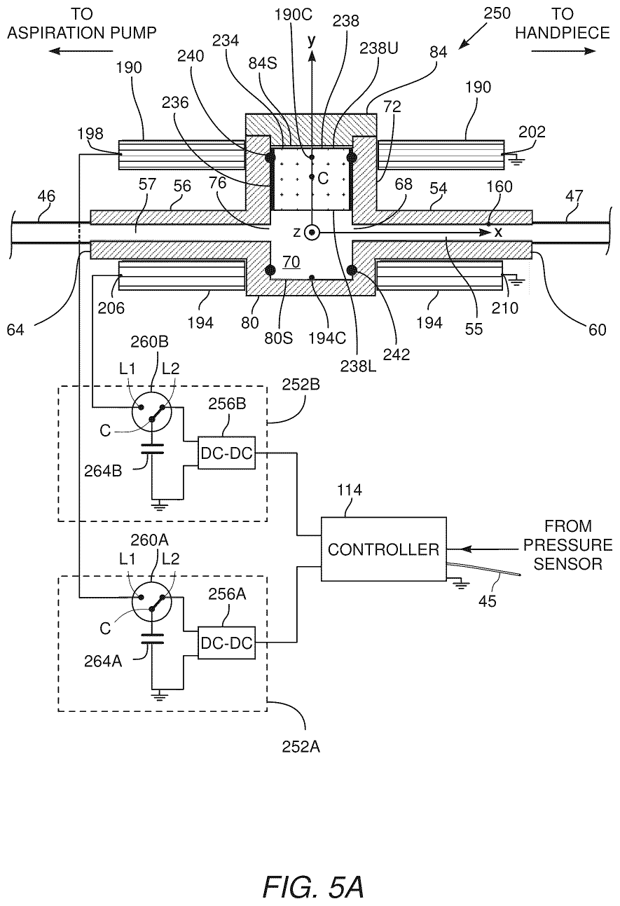

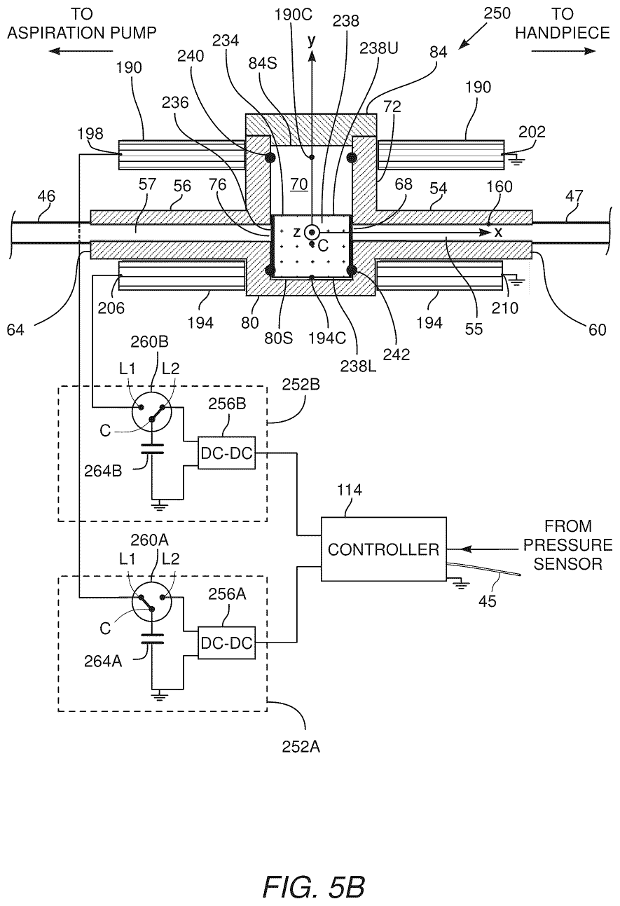

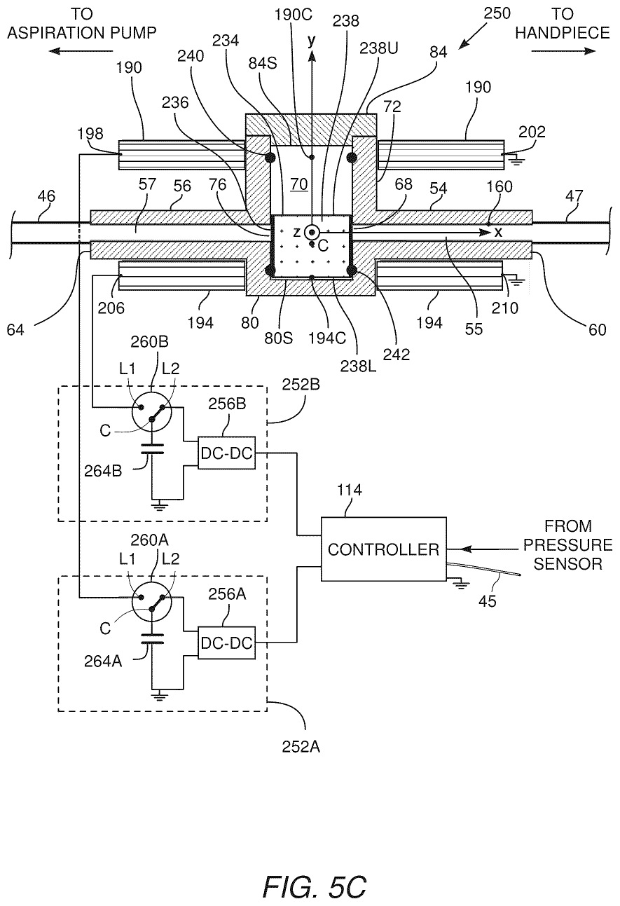

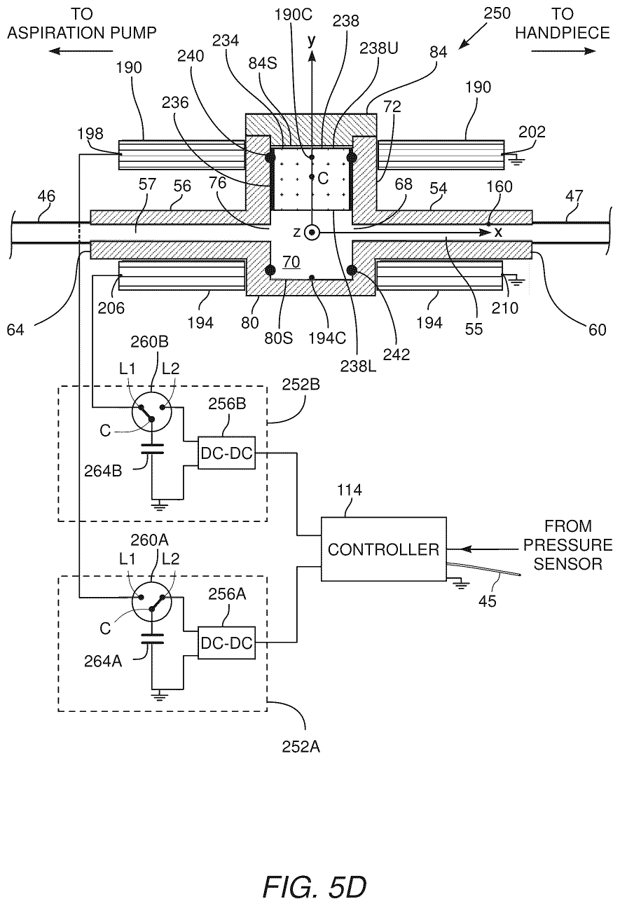

FIGS. 5A, 5B, 5C, and 5D are schematic cross-sections of an AVSS 250, also herein termed valve 250, used in apparatus 11, according to a further alternative example of the present disclosure. Apart from the differences described below, the operation of AVSS 250 is generally similar to that of AVSS 150 (FIGS. 3, 4A, 4B), and elements indicated by the same reference numerals in AVSS 150 and AVSS 250 are generally similar in construction and in operation.

In the following description at least some elements of valve 250 are assumed to be disposable. For example, pin 234, described below, may disposed of and replaced by a new pin. However, in other examples valve 250 may be configured to be reusable. Valve 250 acts to open and close tubing line 46.

FIGS. 5A and 5D illustrate AVSS 250 in its open state wherein aspirated eye fluid may transfer between lumens 55 and 57, and FIGS. 5B and 5C illustrate the AVSS in its closed state, wherein aspirated eye fluid is prevented from transfer between the lumens.

In AVSS 250, in contrast to AVSS 150 and AVSS 50, DC magnetic fields are not generated in coils 190 and 194, for the purpose of translating, and maintaining in place, a pin 234 of AVSS 250. (Pin 234 is described below, and in AVSS 150 and AVSS 50 the DC magnetic fields are generated by local controller 114 applying DC currents to coils 190 and 194.) Rather, in AVSS 250, local controller 114 is configured to apply an electric pulse to each of coils 190 and 194 by operating a driving circuit 252A coupled with coil 190, and a driving circuit 252B coupled with coil 194, the pulses generating corresponding pulsing magnetic fields.

Circuits 252A and 252B are substantially similar to each other and are herein generically termed circuit 252. Each circuit 252 comprises a DC-DC converter 256A, 256B, generically termed converter 256, that is coupled via a single pole double throw (SPDT) switch 260A, 260B, generically termed switch 260, to a capacitor 264A, 264B, generically termed capacitor 264. DC-DC converter 256 receives a low voltage from controller 114, and, under control of the controller 114, converts the low voltage to a high voltage. In a disclosed example the high voltage value is in the range of approximately 1 kV to approximately 2 kV.

The SPDT switch has three poles: a common pole C, and two switchable poles L1, L2. In a charging state of circuit 252, as shown for both circuits 252A and 252B in FIG. 5A and FIG. 5C, pole C of switch 260A is connected to pole L2 of the switch, so that capacitors 264A and 264B charge. A discharging state of circuit 252 is shown in FIG. 5B for circuit 252A and in FIG. 5D for circuit 252B. In FIG. 5B pole C of switch 260A is connected to pole L1 of the switch, so that capacitor 264A discharges through its connected coil, coil 190. In FIG. 5D pole C of switch 260B is connected to pole L1 of the switch, so that capacitor 264B discharges through coil 194.

Because each coil 190 and 194 has a low resistance, typically 0.1Ω or less, the capacitor discharge through its respective coil results in a large pulsatory current, typically in an approximate range of 10-20 amps or more, producing a correspondingly large magnetic field pulse.

In AVSS 250 a cylindrical pin 234 has similar dimensions to those of pin 134 of AVSS 50, described above, has the y-axis as its axis of symmetry, and is configured to slide along the y-axis in lumen 70. However, while pin 234 is conductive, it is not ferromagnetic, unlike pin 134. Pin 234 may be formed as a solid cylinder, as a hollow open-ended unfilled cylinder, or as a hollow cylinder which is filled with a non-conductive material. Other shapes of the pin 234 and lumen 70 are envisioned that would still provide the desired operation of the valve.

In the description herein, pin 234 is, by way of example, assumed to comprise a hollow conductive cylinder 236 filled with a non-conductive material 238, such as a plastic, and those having ordinary skill in the art will be able to adapt the description, mutatis mutandis, for case when pin 234 is solid or as an unfilled cylinder.

During operation of AVSS 250 pin 234 is in one of two stable positions: an open position, corresponding to the open state of the AVSS 250, wherein the pin 234 is proximate to coil 190, as illustrated in FIG. 5A and FIG. 5D, and a closed position, corresponding to the closed state of the AVSS 250, wherein in the pin 234 is proximate to coil 194, as illustrated in FIG. 5B and FIG. 5C. The two stable positions of pin 234, and the transition between the positions, are described further below. In the upper open position, an upper surface 238U of material 238 of pin 234 is assumed to contact a lower surface 84S of stopper 84. In the lower closed position, a lower surface 238L is assumed to contact an upper surface 80S of termination 80.

As stated above, in AVSS 250 coils 190 and 194 are not energized by DC currents, and so no DC magnetic field is used to retain pin 234 in either its open position or its closed position. Rather, in AVSS 250 friction retains pin 234 in its open position and in its closed position.

In an example of the present disclosure, a compressible O-ring 240 is inserted into the wall of lumen 70, in proximity to coil 190, and friction between the O-ring 240 and pin 234 holds the pin stably in its open position. Similarly, a compressible O-ring 242 is inserted into the wall of lumen 70 in proximity to coil 194, and friction between the O-ring 242 and pin 234 holds the pin stably in its closed position.

It will be understood that the O-rings described above are but one type of friction element that may be used to maintain pin 234 in its open and closed positions, and other friction elements, for example configuring protuberances in surfaces 84s and 80S, and mating recesses in surfaces 238U and 238L of material 238, may be used. All such friction elements are assumed to be comprised in the scope of the present disclosure.

The discharge of capacitor 264 through its connected coil generates a pulsing magnetic field through the coil. When pin 234 is proximate to the coil, eddy currents are generated in cylinder 236 of the pin, the eddy currents generating a countervailing pulsing magnetic field. Except for the case when cylinder 236 is positioned symmetrically with respect to the coil, in which case no force is exerted, the generated countervailing magnetic field provides a force directed to repel the pin from the coil.

Examples of the present disclosure use this repulsion property to facilitate the operation of AVSS 250.

Thus, as illustrated in FIG. 5A, coil 190 is located so that it is proximate to stopper 84, and so that a center 190C of the coil 190 is above a center C of cylinder 236 when pin 234 is in its open position. It will be understood that in this arrangement cylinder 236 is asymmetrically disposed with respect to coil 190. Because of the asymmetry the magnetic fields generated by the eddy currents induced in cylinder 236 when capacitor 264A discharges through coil 190, by switch 260A being switched to a discharge state, as shown in FIG. 5B, are in opposition to those generated by the coil. Cylinder 236, and its associated pin 234, is thus repelled to the closed position of the pin, as shown in FIG. 5B.

In the closed position of pin 234, as is also shown in FIG. 5C (which shows both capacitors 264A and 264B as being charged) coil 194 is located to be proximate to termination 80, and so that a center 194C of the coil 194 is below center C of cylinder 236. In this arrangement cylinder 236 is asymmetrically disposed with respect to coil 194. Because of the asymmetry the magnetic fields generated by the eddy currents induced in cylinder 236 when capacitor 264B discharges through coil 194, by switch 260B being switched to a discharge state, as shown in FIG. 5D, are in opposition to those generated by the coil. Cylinder 236, and its associated pin 234, is thus repelled to the open position of the pin 234, as shown in FIG. 5D.

In contrast to AVSS 50 and AVSS 150, controller 114 is not configured to maintain AVSS 250 in its open state or in its closed state; rather, as explained above, friction is used to implement such maintenance. In AVSS 250, when the pressure in the aspiration tubing line 46, as indicated to the controller 114 by the signal from pressure sensor 27, reduces below the preset value, i.e., when the vacuum in the aspiration tubing line increases, or is outside the desired or predetermined range, controller 114 is configured to change the state of AVSS 250 from its open state to its closed state by energizing coil 190 so as to repel pin 234 from its open position to its closed position.

Similarly, when the pressure in the aspiration tubing line 46, as indicated to the controller 114 by the signal from pressure sensor 27, increases above the preset value or is within the desired or predetermined range, controller 114 is configured to change the state of AVSS 250 from its closed state to its open state by energizing coil 194 so as to repel pin 234 from its closed position to its open position.

While the description above of AVSS 250 has assumed that coils 190 and 194 are formed from PCBs, in some examples of AVSS 250 at least one of the coils may be formed from single wires.

### EXAMPLES

Example 1. A phacoemulsification valve (50) for controlling flow of eye fluid, comprising: an enclosure (72); a first lumen (70) within the enclosure, having a first termination (80) and a second termination (84) opposite the first termination; a second lumen (55, 57) within the enclosure, intersecting the first lumen, so that the first lumen separates the second lumen into a first section (54) and a second section (56); a pin (134), configured to slide within the first lumen between a first position, proximate to the first termination, wherein the pin prevents transfer of the eye fluid between the first section and the second section and a second position, proximate to the second termination, wherein the pin permits transfer of the eye fluid between the first section and the second section; a first coil (94), positioned in proximity to the first termination, which is configured when energized to transfer the pin between the first position and the second position; and a second coil (90), positioned in proximity to the second termination, which is configured when energized to transfer the pin between the second position and the first position.

Example 2. The phacoemulsification valve according to example 1, wherein at least one of the first coil and the second coil is formed as a single wire.

Example 3. The phacoemulsification valve according to example 1, wherein at least one of the first coil and the second coil is formed from a printed circuit board.

Example 4. The phacoemulsification valve according to example 1, wherein the pin is formed as a solid cylinder.

Example 5. The phacoemulsification valve according to example 1, wherein the pin is formed as a hollow open-ended cylinder.

Example 6. The phacoemulsification valve according to example 5, wherein the hollow open-ended cylinder is filled with a non-conductive material.

Example 7. The phacoemulsification valve according to any of examples 1 to 6, further comprising a first friction element (240) configured to maintain the pin in the first position, and a second friction element (242) configured to maintain the pin in the second position.

Example 8. The phacoemulsification valve according to example 7, wherein at least one of the first friction element and the second friction element comprises an o-ring.

Example 9. The phacoemulsification valve according to any of examples 1 to 8, wherein the pin in its first position is disposed asymmetrically with respect to the first coil, and wherein the pin in its second position is disposed asymmetrically with respect to the second coil.

Example 10. The phacoemulsification valve according to any of examples 1 to 9, wherein the pin has a pin-center, the first coil has a first coil-center, and the second coil has a second coil-center, and wherein in the first and the second positions the pin center is between the first coil-center and the second coil-center.

Example 11. The phacoemulsification valve according to any of examples 1 to 10, further comprising a controller (114) configured to activate a first circuit (252A) to energize the first coil with a first electric pulse, and to activate a second circuit (252B) to energize the second coil with a second electric pulse.

Example 12. The phacoemulsification valve according to example 11, wherein the first circuit comprises a first voltage generator coupled via a first switch with a first capacitor, and the second circuit comprises a second voltage generator coupled via a second switch with a second capacitor, and wherein the controller is configured to charge the first capacitor via the first switch, and to discharge the first capacitor, when charged, through the first coil via the first switch, and to charge the second capacitor via the second switch, and to discharge the second capacitor, when charged, through the second coil via the second switch.

Example 13. The phacoemulsification valve according to any of examples 1 to 12, further comprising a pressure sensor (27), configured to provide an indication of a pressure within an aspiration channel or an aspiration tubing line coupled with the second lumen, and wherein the controller is configured to discharge one of the first capacitor and the second capacitor in response to the indication.

Example 14. The phacoemulsification valve according to any of examples 1 to 13, wherein the pin comprises a conductive, non-ferromagnetic pin, and wherein the first coil surrounds the first termination and when energized repels the pin from the first position to the second position, and wherein the second coil surrounds the second termination and when energized repels the pin from the second position to the first position.

Example 15. The phacoemulsification valve according to any of examples 1 to 14, wherein the pin comprises a ferromagnetic pin, and wherein the first coil when energized attracts the pin from the second position to the first position, and wherein the second coil when energized attracts the pin from the first position to the second position.

Example 16. The phacoemulsification valve according to any of examples 1 to 15, and comprising a controller configured to energize the first coil with a direct current (DC), and to energize the second coil with the DC current.

Example 17. The phacoemulsification valve according to any of examples 1 to 16, wherein the first termination comprises a stopper removable from the first lumen, and wherein the pin is removable from the first lumen after removal of the stopper.

Example 18. The phacoemulsification valve according to any of examples 1 to 17, wherein the pin is non-sterilizable.

Example 19. The phacoemulsification valve according to any of examples 1 to 18, wherein the enclosure, the first lumen, the second lumen, the first coil, and the second coil are sterilizable.

Example 20. A phacoemulsification apparatus (10), comprising: a phacoemulsification handpiece (12) comprising an aspiration channel (47) configured to transfer eye fluid from an eye of a patient; an aspiration tubing line (46) having a proximal end and a distal end, wherein the distal end is coupled with the aspiration channel; a pressure sensor (27), coupled with the aspiration channel, configured to provide an indication of a pressure within the aspiration channel or the aspiration tubing line; a valve (50) comprising: a first coil (94); a second coil (90); a first lumen (55)) and a second lumen (57) collinear with the first lumen, the first lumen and the second lumen being located between the first coil and the second coil, and being configured to permit transfer of the eye fluid therebetween, the first lumen having a termination coupled with the proximal end of the aspiration tubing line; and a pin (134) configured, when the first coil is energized, to reside at a location between the first and the second lumen so as to prevent the eye fluid from transferring therebetween, and, when the second coil is energized, to translate from the location so as to permit the eye fluid transferring therebetween; and a controller (114), configured to receive the indication from the pressure sensor, and in response, energize one of the first coil and the second coil.

Example 21. The phacoemulsification apparatus according to example 20, further comprising a third lumen, intersecting the first and the second lumens at the location, and wherein the pin is configured to translate within the third lumen.

Example 22. The phacoemulsification apparatus according to any of examples 20 to 21, wherein the first coil surrounds the third lumen, and wherein the second coil surrounds the third lumen.

Example 23. The phacoemulsification apparatus according to any of examples 20 to 22, wherein at least one of the first coil and the second coil is formed from a single wire.

Example 24. The phacoemulsification apparatus according to any of examples 20 to 23, wherein at least one of the first coil and the second coil is formed from a printed circuit board.

Example 25. The phacoemulsification apparatus according to any of examples 20 to 24, wherein the first coil is located at a first position proximate to the location, and the second coil is located at a second position separate from the first position, and wherein energizing the first coil comprises energizing the first coil with a direct current (DC) so as to attract the pin to the location, and energizing the second coil comprises energizing the second coil with the DC current so as to attract the pin from the location.

Example 26. The phacoemulsification apparatus according to any of examples 20 to 25, wherein the first coil is located at a first position proximate to the location, and the second coil is located at a second position separate from the first position, and wherein energizing the first coil comprises energizing the first coil with a current pulse so as to repel the pin from the location, and energizing the second coil comprises energizing the second coil with the current pulse so as to repel the pin to the location.

Example 27. The phacoemulsification apparatus according to any of examples 20 to 26, wherein the pin comprises a conductive, non-ferromagnetic pin.

Example 28. The phacoemulsification apparatus according to any of examples 20 to 27, wherein the pin comprises a ferromagnetic pin.

Example 29. A method for controlling eye fluid, comprising: providing an enclosure (72); forming a first lumen (70) within the enclosure, the first lumen having a first termination (80) and a second termination (84) opposite the first termination; forming a second lumen (55, 57) within the enclosure, to intersect the first lumen, so that the first lumen separates the second lumen into a first section (54) and a second section (56); configuring a pin (134) to slide within the first lumen between a first position, proximate to the first termination, wherein the pin prevents transfer of the eye fluid between the first section and the second section, and a second position, proximate to the second termination, wherein the pin permits transfer of the eye fluid between the first section and the second section; configuring a first coil (94), positioned in proximity to the first termination, when energized, to transfer the pin between the first position and the second position; and configuring a second coil (90), positioned in proximity to the second termination, when energized, to transfer the pin between the second position and the first position.

Example 30. A method, comprising: providing a phacoemulsification handpiece (12) comprising an aspiration channel (47) configured to transfer eye fluid from an eye of a patient; coupling a pressure sensor (27) with the aspiration channel, and configuring the pressure sensor to provide an indication of a pressure within the aspiration channel or an aspiration tubing line; coupling a distal end of the aspiration tubing line to the aspiration channel, the aspiration tubing line having a proximal end, providing a valve (50) comprising: a first coil (94); a second coil (90); a first lumen (55) and a second lumen (57) collinear with the first lumen, the first and the second lumens being located between the first coil and the second coil, and being configured to transfer the eye fluid therebetween, the first lumen having a termination coupled to the proximal end of the aspiration tubing line; and a pin (134) configured, when the first coil is energized, to reside at a location between the first lumen and the second lumen so as to prevent the eye fluid from transferring therebetween, and, when the second coil is energized, to translate from the location so as to permit the eye fluid transferring therebetween; and receiving the indication from the pressure sensor, and in response, energizing one of the first coil and the second coil.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A phacoemulsification valve for controlling flow of eye fluid, comprising:
an enclosure;
a first lumen within the enclosure, having a first termination and a second termination opposite the first termination;
a second lumen within the enclosure, intersecting the first lumen, so that the first lumen separates the second lumen into a first section and a second section;
a pin, configured to slide within the first lumen between a first position, proximate to the first termination, wherein the pin prevents transfer of the eye fluid between the first section and the second section and a second position, proximate to the second termination, wherein the pin permits transfer of the eye fluid between the first section and the second section;
a first coil, positioned in proximity to the first termination, which is configured when energized to transfer the pin between the first position and the second position; and
a second coil, positioned in proximity to the second termination, which is configured when energized to transfer the pin between the second position and the first position.

2. The phacoemulsification valve according to claim 1, wherein at least one of the first coil and the second coil is formed as a single wire.

3. The phacoemulsification valve according to claim 1, wherein at least one of the first coil and the second coil is formed from a printed circuit board.

4. The phacoemulsification valve according to claim 1, wherein the pin is formed as a solid cylinder.

5. The phacoemulsification valve according to claim 1, wherein the pin is formed as a hollow open-ended cylinder.

6. The phacoemulsification valve according to claim 5, wherein the hollow open-ended cylinder is filled with a non-conductive material.

7. The phacoemulsification valve according to claim 1, further comprising a first friction element configured to maintain the pin in the first position, and a second friction element configured to maintain the pin in the second position.

8. The phacoemulsification valve according to claim 7, wherein at least one of the first friction element and the second friction element comprises an o-ring.

9. The phacoemulsification valve according to claim 1, wherein the pin in its first position is disposed asymmetrically with respect to the first coil, and wherein the pin in its second position is disposed asymmetrically with respect to the second coil.

10. The phacoemulsification valve according to claim 9, wherein the pin has a pin-center, the first coil has a first coil-center, and the second coil has a second coil-center, and wherein in the first and the second positions the pin center is between the first coil-center and the second coil-center.

11. The phacoemulsification valve according to claim 1, further comprising a controller configured to activate a first circuit to energize the first coil with a first electric pulse, and to activate a second circuit to energize the second coil with a second electric pulse.

12. The phacoemulsification valve according to claim 11, wherein the first circuit comprises a first voltage generator coupled via a first switch with a first capacitor, and the second circuit comprises a second voltage generator coupled via a second switch with a second capacitor, and wherein the controller is configured to charge the first capacitor via the first switch, and to discharge the first capacitor, when charged, through the first coil via the first switch, and to charge the second capacitor via the second switch, and to discharge the second capacitor, when charged, through the second coil via the second switch.

13. The phacoemulsification valve according to claim 12, further comprising a pressure sensor, configured to provide an indication of a pressure within an aspiration channel or an aspiration tubing line coupled with the second lumen, and wherein the controller is configured to discharge one of the first capacitor and the second capacitor in response to the indication.

14. The phacoemulsification valve according to claim 1, wherein the pin comprises a conductive, non-ferromagnetic pin, and wherein the first coil surrounds the first termination and when energized repels the pin from the first position to the second position, and wherein the second coil surrounds the second termination and when energized repels the pin from the second position to the first position.

15. The phacoemulsification valve according to claim 1, wherein the pin comprises a ferromagnetic pin, and wherein the first coil when energized attracts the pin from the second position to the first position, and wherein the second coil when energized attracts the pin from the first position to the second position.

16. The phacoemulsification valve according to claim 1, and comprising a controller configured to energize the first coil with a direct current (DC), and to energize the second coil with the DC current.

17. The phacoemulsification valve according to claim 1, wherein the first termination comprises a stopper removable from the first lumen, and wherein the pin is removable from the first lumen after removal of the stopper.

18. The phacoemulsification valve according to claim 1, wherein the pin is non-sterilizable.

19. The phacoemulsification valve according to claim 1, wherein the enclosure, the first lumen, the second lumen, the first coil, and the second coil are sterilizable.

20. A phacoemulsification apparatus, comprising:
a phacoemulsification handpiece comprising an aspiration channel configured to transfer eye fluid from an eye of a patient;
an aspiration tubing line having a proximal end and a distal end, wherein the distal end is coupled with the aspiration channel;
a pressure sensor, coupled with the aspiration channel, configured to provide an indication of a pressure within the aspiration channel or the aspiration tubing line;
a valve comprising:
a first coil;
a second coil;
a first lumen and a second lumen collinear with the first lumen, the first lumen and the second lumen being located between the first coil and the second coil, and being configured to permit transfer of the eye fluid therebetween, the first lumen having a termination coupled with the proximal end of the aspiration tubing line; and
a pin configured, when the first coil is energized, to reside at a location between the first and the second lumen so as to prevent the eye fluid from transferring therebetween, and, when the second coil is energized, to translate from the location so as to permit the eye fluid transferring therebetween; and
a controller, configured to receive the indication from the pressure sensor, and in response, energize one of the first coil and the second coil.

21. The phacoemulsification apparatus according to claim 20, further comprising a third lumen, intersecting the first and the second lumens at the location, and wherein the pin is configured to translate within the third lumen.

22. The phacoemulsification apparatus according to claim 21, wherein the first coil surrounds the third lumen, and wherein the second coil surrounds the third lumen.

23. The phacoemulsification apparatus according to claim 20, wherein at least one of the first coil and the second coil is formed from a single wire.

24. The phacoemulsification apparatus according to claim 20, wherein at least one of the first coil and the second coil is formed from a printed circuit board.

25. The phacoemulsification apparatus according to claim 20, wherein the first coil is located at a first position proximate to the location, and the second coil is located at a second position separate from the first position, and wherein energizing the first coil comprises energizing the first coil with a direct current (DC) so as to attract the pin to the location, and energizing the second coil comprises energizing the second coil with the DC current so as to attract the pin from the location.

26. The phacoemulsification apparatus according to claim 20, wherein the first coil is located at a first position proximate to the location, and the second coil is located at a second position separate from the first position, and wherein energizing the first coil comprises energizing the first coil with a current pulse so as to repel the pin from the location, and energizing the second coil comprises energizing the second coil with the current pulse so as to repel the pin to the location.

27. The phacoemulsification apparatus according to claim 20, wherein the pin comprises a conductive, non-ferromagnetic pin.

28. The phacoemulsification apparatus according to claim 20, wherein the pin comprises a ferromagnetic pin.

29. A method for controlling eye fluid, comprising:
providing an enclosure;
forming a first lumen within the enclosure, the first lumen having a first termination and a second termination opposite the first termination;
forming a second lumen within the enclosure, to intersect the first lumen, so that the first lumen separates the second lumen into a first section and a second section;
configuring a pin to slide within the first lumen between a first position, proximate to the first termination, wherein the pin prevents transfer of the eye fluid between the first section and the second section, and a second position, proximate to the second termination, wherein the pin permits transfer of the eye fluid between the first section and the second section;
configuring a first coil, positioned in proximity to the first termination, when energized, to transfer the pin between the first position and the second position; and
configuring a second coil, positioned in proximity to the second termination, when energized, to transfer the pin between the second position and the first position.

30. The method according to claim 29, wherein at least one of the first coil and the second coil is formed as a single wire.

31. The method according to claim 29, wherein at least one of the first coil and the second coil is formed from a printed circuit board.

32. The method according to claim 29, wherein the pin is formed as a solid cylinder.

33. The method according to claim 29, wherein the pin is formed as a hollow open-ended cylinder.

34. The method according to claim 33, wherein the hollow open-ended cylinder is filled with a non-conductive material.

35. The method according to claim 29, further comprising configuring a first friction element to maintain the pin in the first position, and configuring a second friction element to maintain the pin in the second position.

36. The method according to claim 35, wherein at least one of the first friction element and the second friction element comprises an o-ring.

37. The method according to claim 29, further comprising disposing the pin in its first position asymmetrically with respect to the first coil, and disposing the pin in its second position asymmetrically with respect to the second coil.

38. The method according to claim 37, wherein the pin has a pin-center, the first coil has a first coil-center, and the second coil has a second coil-center, and wherein in the first and the second positions the pin center is between the first coil-center and the second coil-center.

39. The method according to claim 29, further comprising activating a first circuit to energize the first coil with a first electric pulse, and activating a second circuit to energize the second coil with a second electric pulse.

40. The method according to claim 39, wherein the first circuit comprises a first voltage generator coupled via a first switch with a first capacitor, and the second circuit comprises a second voltage generator coupled via a second switch with a second capacitor, the method further comprising charging the first capacitor via the first switch, and discharging the first capacitor, when charged, through the first coil via the first switch, and charging the second capacitor via the second switch, and discharging the second capacitor, when charged, through the second coil via the second switch.

41. The method according to claim 40, further comprising configuring a pressure sensor to provide an indication of a pressure within an aspiration channel or an aspiration tubing line coupled with the second lumen, and discharging one of the first capacitor and the second capacitor in response to the indication.

42. The method according to claim 29, wherein the pin comprises a conductive, non-ferromagnetic pin, and wherein the first coil surrounds the first termination and when energized repels the pin from the first position to the second position, and wherein the second coil surrounds the second termination and when energized repels the pin from the second position to the first position.

43. The method according to claim 29, wherein the pin comprises a ferromagnetic pin, and wherein the first coil when energized attracts the pin from the second position to the first position, and wherein the second coil when energized attracts the pin from the first position to the second position.

44. The method according to claim 29, and comprising energizing the first coil with a direct current (DC), and energizing the second coil with the DC current.

45. The method according to claim 29, wherein the first termination comprises a stopper removable from the first lumen, and wherein the pin is removable from the first lumen after removal of the stopper.

46. The method according to claim 29, wherein the pin is non-sterilizable.

47. The method according to claim 29, wherein the enclosure, the first lumen, the second lumen, the first coil, and the second coil are sterilizable.

48. A method, comprising:
providing a phacoemulsification handpiece comprising an aspiration channel configured to transfer eye fluid from an eye of a patient;
coupling a pressure sensor with the aspiration channel, and configuring the pressure sensor to provide an indication of a pressure within the aspiration channel or an aspiration tubing line;
coupling a distal end of the aspiration tubing line to the aspiration channel, the aspiration tubing line having a proximal end,
providing a valve comprising:
a first coil;
a second coil;
a first lumen and a second lumen collinear with the first lumen, the first and the second lumens being located between the first coil and the second coil, and being configured to transfer the eye fluid therebetween, the first lumen having a termination coupled to the proximal end of the aspiration tubing line; and
a pin configured, when the first coil is energized, to reside at a location between the first lumen and the second lumen so as to prevent the eye fluid from transferring therebetween, and, when the second coil is energized, to translate from the location so as to permit the eye fluid transferring therebetween; and
receiving the indication from the pressure sensor, and in response, energizing one of the first coil and the second coil.

49. The method according to claim 48, further comprising forming a third lumen to intersect the first lumen and the second lumen at the location, and configuring the pin to translate within the third lumen.

50. The method according to claim 49, wherein the first coil surrounds the third lumen, and wherein the second coil surrounds the third lumen.

51. The method according to claim 48, wherein at least one of the first coil and the second coil is formed from a single wire.

52. The method according to claim 48, wherein at least one of the first coil and the second coil is formed from a printed circuit board.

53. The method according to claim 48, wherein the first coil is located at a first position proximate to the location, and the second coil is located at a second position separate from the first position, and wherein energizing the first coil comprises energizing the first coil with a direct current (DC) so as to attract the pin to the location, and energizing the second coil comprises energizing the second coil with the DC current so as to attract the pin from the location.

54. The method according to claim 48, wherein the first coil is located at a first position proximate to the location, and the second coil is located at a second position separate from the first position, and wherein energizing the first coil comprises energizing the first coil with a current pulse so as to repel the pin from the location, and energizing the second coil comprises energizing the second coil with the current pulse so as to repel the pin to the location.

55. The method according to claim 48, wherein the pin comprises a conductive, non-ferromagnetic pin.

56. The method according to claim 48, wherein the pin comprises a ferromagnetic pin.
